SEQUENCE LISTING (48 sequences)

SEQ ID NO: 1 - DNA, 20 bp, Artificial Sequence
  The sequence is synthetized
tccctcaagattgtcagcaa

SEQ ID NO: 2 - DNA, 18 bp, Artificial Sequence
  The sequence is synthetized
agatccacaacggataca

SEQ ID NO: 3 - DNA, 20 bp, Artificial Sequence
  The sequence is synthetized
tcctggagaaagatggcgac

SEQ ID NO: 4 - DNA, 20 bp, Artificial Sequence
  The sequence is synthetized
ttcactttgccgaaggtgcc

SEQ ID NO: 5 - DNA, 20 bp, Artificial Sequence
  The sequence is synthetized
tgaaggctgtggtgatggat

SEQ ID NO: 6 - DNA, 20 bp, Artificial Sequence
  The sequence is synthetized
ccgtagtggttgaggttgga

SEQ ID NO: 7 - DNA, 24 bp, Artificial Sequence
  The sequence is synthetized
gcgatatgctgtggtgcttaatgc

SEQ ID NO: 8 - DNA, 23 bp, Artificial Sequence
  The sequence is synthetized
ggagtggtggtagttgtggaagc

SEQ ID NO: 9 - DNA, 20 bp, Artificial Sequence
  The sequence is synthetized
tacatcgactgcatcaggca

SEQ ID NO: 10 - DNA, 17 bp, Artificial Sequence
  The sequence is synthetized
gatactttcccgtcgca

SEQ ID NO: 11 - DNA, 22 bp, Artificial Sequence
  The sequence is synthetized
actgcagtgtaggactttcctg

SEQ ID NO: 12 - DNA, 20 bp, Artificial Sequence
  The sequence is synthetized
ccggtcacagcccatttttc

SEQ ID NO: 13 - DNA, 20 bp, Artificial Sequence
  The sequence is synthetized
gctgatgacggctatggtgt

SEQ ID NO: 14 - DNA, 20 bp, Artificial Sequence
  The sequence is synthetized
gtccatggtctcctccaagg

SEQ ID NO: 15 - DNA, 20 bp, Artificial Sequence
  The sequence is synthetized
cgagagtcagcctttaacga

SEQ ID NO: 16 - DNA, 20 bp, Artificial Sequence
  The sequence is synthetized
agatgcaggctccactttga

SEQ ID NO: 17 - DNA, 18 bp, Artificial Sequence
  The sequence is synthetized
ggcacgggaggatggact

SEQ ID NO: 18 - DNA, 22 bp, Artificial Sequence
  The sequence is synthetized
gcttctttgctgtgagatgacc

SEQ ID NO: 19 - DNA, 24 bp, Artificial Sequence
  The sequence is synthetized
tcctcggtgacttatcctgtggtc

SEQ ID NO: 20 - DNA, 23 bp, Artificial Sequence
  The sequence is synthetized
gcgtggactccgtaatgatagcc

SEQ ID NO: 21 - DNA, 21 bp, Artificial Sequence
  The sequence is synthetized
gctcctactggcctgaggttg

SEQ ID NO: 22 - DNA, 20 bp, Artificial Sequence
  The sequence is synthetized
tgctgcggaaagaacaaggc

SEQ ID NO: 23 - DNA, 20 bp, Artificial Sequence
  The sequence is synthetized
ggaaagggcttggtggtaca

SEQ ID NO: 24 - DNA, 20 bp, Artificial Sequence
  The sequence is synthetized
tggctctgacacatgcacac

SEQ ID NO: 25 - DNA, 20 bp, Artificial Sequence
  The sequence is synthetized
cagaccccacctcaaacaca

SEQ ID NO: 26 - DNA, 20 bp, Artificial Sequence
  The sequence is synthetized
aatccctgctttcctgccaa

SEQ ID NO: 27 - DNA, 20 bp, Artificial Sequence
  The sequence is synthetized
gccatgaagtcccttagcct

SEQ ID NO: 28 - DNA, 20 bp, Artificial Sequence
  The sequence is synthetized
ggcttcagggttcctagtct

SEQ ID NO: 29 - DNA, 19 bp, Artificial Sequence
  The sequence is synthetized
gttgtctcctgcgacttca

SEQ ID NO: 30 - DNA, 19 bp, Artificial Sequence
  The sequence is synthetized
gcccctcctgttattatgg

SEQ ID NO: 31 - DNA, 20 bp, Artificial Sequence
  The sequence is synthetized
atgggacaagccttgctatg

SEQ ID NO: 32 - DNA, 20 bp, Artificial Sequence
  The sequence is synthetized
ctcagtcatgggcacaaatg

SEQ ID NO: 33 - DNA, 20 bp, Artificial Sequence
  The sequence is synthetized
gaagggtgctaaacccgaaa

SEQ ID NO: 34 - DNA, 22 bp, Artificial Sequence
  The sequence is synthetized
tctgctccctatatccgttctt

SEQ ID NO: 35 - DNA, 23 bp, Artificial Sequence
  The sequence is synthetized
tggggctgctaatctctgggtgt

SEQ ID NO: 36 - DNA, 24 bp, Artificial Sequence
  The sequence is synthetized
ggctttatctctggggtgggtttg

SEQ ID NO: 37 - DNA, 20 bp, Artificial Sequence
  The sequence is synthetized
aagttgcccgagtcagagaa

SEQ ID NO: 38 - DNA, 20 bp, Artificial Sequence
  The sequence is synthetized
cgtcgaacttggagagatcc

SEQ ID NO: 39 - DNA, 18 bp, Artificial Sequence
  The sequence is synthetized
tggtcaagcagctagaaa

SEQ ID NO: 40 - DNA, 19 bp, Artificial Sequence
  The sequence is synthetized
cccagtctcatttagccac

SEQ ID NO: 41 - DNA, 19 bp, Artificial Sequence
  The sequence is synthetized
gactccgctcgctcattcc

SEQ ID NO: 42 - DNA, 20 bp, Artificial Sequence
  The sequence is synthetized
ggcagatctgtttgagggct

SEQ ID NO: 43 - DNA, 21 bp, Artificial Sequence
  The sequence is synthetized
gtgccagtttcgatccgtaga

SEQ ID NO: 44 - DNA, 20 bp, Artificial Sequence
  The sequence is synthetized
ggccagcatcgtgtagatga

SEQ ID NO: 45 - DNA, 20 bp, Artificial Sequence
  The sequence is synthetized
ctggtgagtggagggaccat

SEQ ID NO: 46 - DNA, 20 bp, Artificial Sequence
  The sequence is synthetized
tgctgcaagaagcggatgta

SEQ ID NO: 47 - DNA, 21 bp, Artificial Sequence
  The sequence is synthetized
tggatggattagattggactg

SEQ ID NO: 48 - DNA, 21 bp, Artificial Sequence
  The sequence is synthetized
tgttgatgagcctgacttcat

(19) United States
(12) Patent Application Publication
(10) Pub. No.: US 2023/0226018 A1
(43) Pub. Date: Jul. 20, 2023

(54) APPLICATION OF COMPOUND USING INTRA-CYCLIC PEROXO-BRIDGED SESQUITERPENES AS PARENT NUCLEUS IN METABOLISM-RELATED FATTY LIVER DISEASES

(71) Applicants: SHANGHAI JIAO TONG UNIVERSITY, Shanghai (CN); SUZHOU TANGJI BIOTECHNOLOGY CO., LTD., Suzhou (CN)

(72) Inventors: Yuliang WANG, Shanghai (CN); Kexuan TANG, Shanghai (CN); Jiangao FAN, Shanghai (CN); Jing ZENG, Shanghai (CN); Xiaofen SUN, Shanghai (CN)

(73) Assignees: SHANGHAI JIAO TONG UNIVERSITY, Shanghai (CN); SUZHOU TANGJI BIOTECHNOLOGY CO., LTD., Suzhou (CN)

(21) Appl. No.: 18/011,198

(22) PCT Filed: Feb. 7, 2021
(86) PCT No.: PCT/CN2021/075714

§ 371 Date: Dec. 19, 2022

(30) Foreign Application Priority Data
Jan. 13, 2021 (CN) 202110041895.3

(51) Int. Cl. A61K 31/357; A61P 1/16

(52) U.S. Cl. CPC A61K 31/357; A61P 1/16

(57) ABSTRACT

An application of a compound using intra-cyclic peroxo-bridged sesquiterpenes as a parent nucleus in metabolism-related fatty liver diseases is provided. The compound using intra-cyclic peroxo-bridged sesquiterpenes as the parent nucleus has the structure shown in Formula I, where A denotes O, —OH, OCH3, —OCH₂CH₃, or —OC₄H₅O₃. The compound using intra-cyclic peroxo-bridged sesquiterpenes as the parent nucleus is configured for preparing a drug or a composition for treating metabolism-related fatty liver diseases. The present invention demonstrates that the compound using intra-cyclic peroxo-bridged sesquiterpenes as the parent nucleus can improve the function of mitochondria. in liver cells and enhance the fatty acid β-oxidation capacity thereof and can be used for the treatment of metabolic dysfunction due to mitochondrial dysfunction, as well as the resulting liver fat accumulation, inflammation, and fibrotic lesions.

### CROSS REFERENCE TO THE RELATED APPLICATIONS

This application is the national phase entry of International Application No. PCT/CN2021/075714, filed on Feb. 7, 2021, which is based upon and claims priority to Chinese Patent Application 202110041895.3, filed on Jan. 13, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBSHJL010_Sequence_Listing.txt, created on Nov. 16, 2022, and is 9,991 bytes in size.

### TECHNICAL FIELD

The present invention relates to the field of drugs for the treatment of liver diseases, in particular, to an application of a compound using intra-cyclic peroxo-bridged sesquiterpenes as a parent nucleus in metabolism-related fatty liver diseases.

### BACKGROUND

Artemisinin compounds, as the most remarkable ones among all compounds with intra-cyclic peroxo-bridged sesquiterpenes in terms of medical application, are currently the first-line drugs for the treatment of malaria. At the same time, many studies have focused on the new efficacy of artemisinin compounds, involving the treatment of tumors, inflammation, asthma, skin diseases, lupus erythematosus, and other diseases. However, except for lupus erythematosus, which has entered the clinical research stage, most new indications only remain in the description of a. phenomenon, and no new indication has been approved for clinical application. The reason is that the above studies mainly focus on pharmacological phenomena and lack mechanisms of action, so it is difficult to find the disease classification for which various compounds are most suitable, and thus the compounds fail to show better effects than the existing drugs.

Precision drug therapy is a category of precision medicine. Precision medicine encompasses precision diagnosis, precision treatment (including precision drug therapy), precision prevention, and the like, and the core concept of precision medicine is to formulate reliable disease prevention and treatment regimens and implement corresponding medical behaviors based on objective and effective indicators and evidence. Traditional clinical treatment focuses on the correlation between empirical evidence, such as medical history and subjective examination, and clinical presentation as well as treatment outcome. Furthermore, traditional clinical treatment pays attention to the group effect and statistical effect, leading to the domination of the concept of group medicine, such as “grouping” and “standardization”. Precision medicine is a personalized medical model where treatment regimens are formulated according to the individual characteristics of patients. The word “precision” is reflected in the precise determination of the causes of individual diseases in combination with precise drug treatment with a. clear mechanism of action. Therefore, the realization of the application of artemisinin compounds in new clinical indications depends on the clarification of the precise mechanism of action of artemisinin compounds in the field of a certain disease, so that the precise therapeutic effect of artemisinin compounds in a certain subdividing spectrum of disease among the certain disease can be confirmed.

### SUMMARY

The present invention aims to solve the current difficulties in the new application of artemisinin drugs and creatively verify its new application in lipid metabolism-related fatty liver diseases, especially the new application in fatty liver diseases caused by mitochondrial dysfunction and decreased fatty acid 1-oxidation capacity. The present invention discloses an application of a compound using infra-cyclic peroxo-bridged sesquiterpenes as a parent nucleus in metabolism-related fatty liver diseases. The compound using intra-cyclic peroxo-bridged sesquiterpenes as the parent nucleus has the structure shown in Formula I:

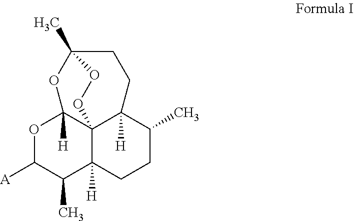

In the Formula, A denotes O, —OH, —OCH₃, —OCH₂CH₃, or —OC₄H₅O₃, and the compound using intra-cyclic peroxo-bridged sesquiterpenes as the parent nucleus is configured for preparing a drug or a composition for treating metabolism-related fatty liver diseases.

Further, when A is —OC₄H₅O₃, the compound using intra-cyclic peroxo-bridged sesquiterpenes as the parent nucleus is artesunate.

Further, when A is O, —OH, —OCH;, or —OCH₂CH₃, the compound using intra-cyclic peroxo-bridged sesquiterpenes as the parent nucleus is artemisinin, dihydroartemisinin, artemether, or arteether, respectively. The chemical structural formulas of artemisinin, artesunate, dihydroartemisinin, artemether, and arteether are as follows:

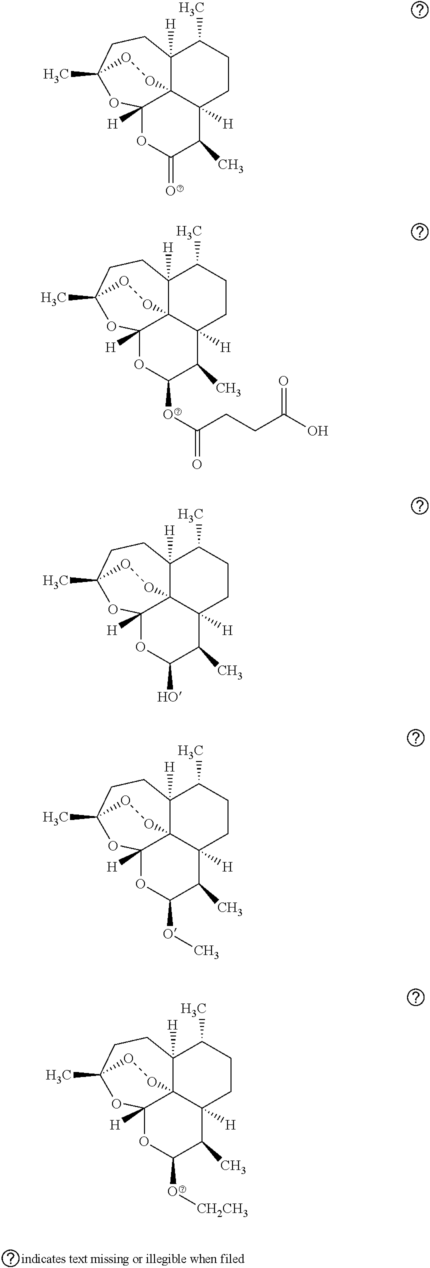

Further, the compound using intra-cyclic peroxo-bridged sesquiterpenes as the parent nucleus has the function of regulating mitochondria in liver cells.

Further, the function of regulating mitochondria in liver cells is to enhance the fatty acid β-oxidation capacity of the mitochondria

Further, metabolism-related fatty liver diseases include liver lesions caused by mitochondrial dysfunction and decreased fatty acid β-oxidation capacity.

Further, the liver lesions include liver steatosis, inflammation, and fibrosis. The present invention also discloses a drug containing the compound with the structure shown in Formula I described above for the prevention or treatment of fatty liver diseases.

Moreover, the present invention discloses a composition for the prevention or treatment of fatty liver diseases, which contains the compound with the structure shown in Formula I described above or a pharmaceutically acceptable salt thereof as an active ingredient.

Further, the composition contains one or more carriers, excipients, or diluents that are pharmaceutically acceptable.

The present invention relates to the application of the compound with the structure shown in Formula I described above or a pharmaceutical salt thereof in the preparation of a product for the prevention or treatment of fatty liver diseases.

Further, fatty liver diseases are liver lesions caused by mitochondrial dysfunction and decreased fatty acid β-oxidation capacity, and the liver lesions include liver steatosis, inflammation, and fibrosis.

The present invention has the following advantages: It is found and proven that a compound using intra-cyclic peroxo-bridged sesquiterpenes as a parent nucleus, in particular, a series of derivatives represented by artemisinin, can improve the function of mitochondria in liver cells and enhance the fatty acid β-oxidation capacity thereof, thereby adapting to the treatment of metabolic dysfunction caused by mitochondrial dysfunction, as well as the resulting liver fat accumulation, inflammation, and fibrotic lesions.

The present invention will be further illustrated in combination with the accompanying drawings to fully explain the purpose, technical features, and technical effects of the present invention.

### BRIEF DESCRIPTION OF THE DRAWINGS

FIG. 1 is a column chart of the content of triglyceride in HepG2 liver cancer cells of a control group, a model group, and an artesunate group.

FIGS. 2A-2B are diagrams of Nile red staining of lipid droplets, where FIG. 2A is the model group, and FIG. 2B is the artesunate group.

FIG. 3 shows the mRNA expression results of CIDE series genes related to lipid droplets. CIDE gene expression is related to the size of lipid droplets in cells. Artesunate can reduce the volume of lipid droplets in cells and the content of fatty acids by reducing CIDE gene expression. In FIGS. 3-7, when each group is compared with the model group, *, **, and *** denote that there are significant differences. Among them, * denotes p<0.05,** denotes p<0.01,*** denotes p<0.001, and **** denotes p<0.0001.

FIG. 4 shows the effect of artesunate on the mRNA expression level of AMPKα. Adenosine 5′-monophosphate (AMP)-dependent protein kinase is a key molecule in regulating bioenergy metabolism. Artesunate increases the expression of AMPKα, indicating that artesunate can activate intracellular energy metabolism and consume excess intracellular fatty acids.

FIG. 5 shows the effect of artesunate on the pathways of adenosine 5′-monophosphate-activated protein kinase (AMPK)-sterol regulatory element binding protein-1c (SREBP1c)-acetyl coA carboxylase (ACC)-stearoyl-coA desaturase-1 (SCDI)-fatty acid synthase (FAS). SREBP1c-ACC/SCD1/FAS is a key upstream gene of intracellular lipid synthesis. Artesunate can reduce the expression of the genes of the pathways, indicating that artesunate can inhibit lipid synthesis in cells and reduce the lipid content in cells.

FIG. 6 shows the effect of artesunate on the pathways of AMPK-cluster of differentiation 36 (CD36)-carnitine palmitoyltransferase 1 (CPT1). The CPT1 gene is a key gene for fatty acids transported to mitochondria; artesunate increases the expression of CPT1, which can accelerate mitochondrial fatty acid oxidation and reduce the lipid content in cells.

FIG. 7 shows the effect of artesunate on the mRNA expressions of PPARα and PPARγ. Artesunate can significantly increase the mRNA expressions of PPARα and PPARγ. Peroxisome proliferator-activated receptor α (PPARα) is an important nuclear receptor that plays an important role in maintaining the homeostasis of body energy metabolism. When a body is in a state of nutrient deficiency, PPARα in the coated plasma will be transferred to the nucleus, and the PPARα entering the nucleus will promote the expression of downstream genes involved in fatty acid oxidation, thereby promoting fatty acid oxidation to ensure the normal energy demand of the body and maintain the homeostasis of liver lipid metabolism.

FIG. 8 shows pathological sections, stained with HE and Oil red O, respectively, of the livers of high-fat and high-sugar-induced mice. Artemisinin can inhibit liver steatosis induced by a high-fat and high-sugar diet.

FIGS. 9A-9H show the effects of artemisinin on serum liver enzymes, lobular inflammation, and ballooning of liver cells. FIG. 9A shows the effect on liver index %. FIG. 9B shows the effect on liver steatosis. FIG. 9C shows the effect on inflammation. FIG. 9D shows the effect on ballooning. FIG. 9E shows the effect on alanine aminotransferase (ALT). FIG. 9F shows the effect on aspartate aminotransferase (AST). FIG. 9G shows the effect on tumor necrosis factor α (TNFα). FIG. 9H shows the effect on interleukin 6 (IL-6). In FIGS. 9A-9H, FIGS. 10A-10I, and FIGS. 11A-11G, Con, HFD, and HFD+Art represent a normal group, a high-fat group, and a high-fat plus artemisinin group, respectively. In FIGS. 9A-9H, FIGS. 10A-10I, and FIGS. 11A-11G, * denotes p<0.05, ** denotes p<0.01, *** denotes p<0.001, and **** denotes p<0.0001.

FIGS. 10A-10I show the expression levels of lipid metabolism-related genes in mice liver; artemisinin intervention can reduce the expression of lipid metabolism-related genes in mice liver, thereby reducing the synthesis of lipids in the liver.

FIGS. 11A-11G show the expression of autophagy-related proteins in mice liver; artemisinin intervention can restore the level of mitophagy in mice to maintain the quantity and quality of mitochondria in mice liver at the optimal state and inhibit mitochondrial damage and liver lesions induced by high-fat and high-sugar. LC3 represents autophagy microtubule-associated protein light chain 3, and TOR represents the rapamycin target protein.

### DETAILED DESCRIPTION OF THE EMBODIMENTS

The technical solution of the present invention is further described by specific embodiments as follows. The following embodiments are further descriptions of the present invention rather than limiting the scope of the present invention. The English abbreviations involved in the present invention are presented in Table 3 at the end of the text.

### Embodiment 1

HepG2 liver cancer cells (purchased from the Cell Bank of the Chinese Academy of Sciences) were selected and used as research materials. HepG2 liver cancer cells are widely used in pharmacological and toxicological studies. They can express many differentiated liver functions, such as plasma protein synthesis and secretion, cholesterol and triglyceride metabolism, lipoprotein metabolism and transport, bile acid synthesis, glycogen synthesis, or insulin signal transduction.

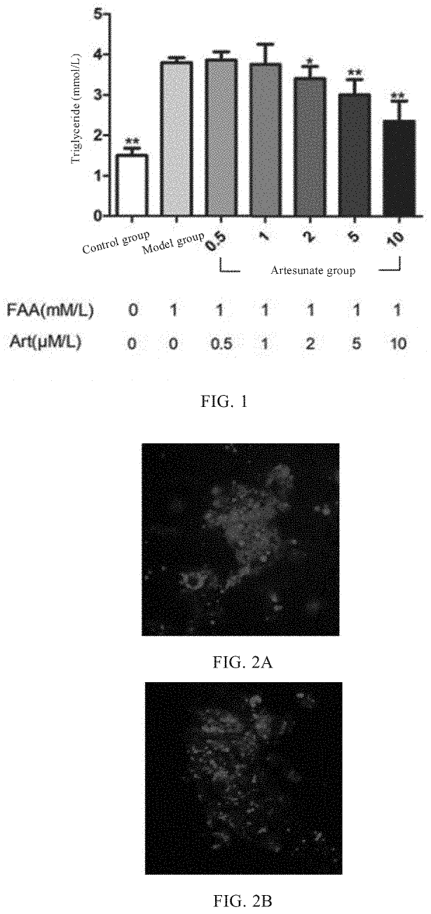

The addition of free fatty acid (FFA) in a medium can cause the accumulation of lipids in HepG2 liver cancer cells, damage the mitochondrial function, and reduce the fatty acid β-oxidation capacity, thereby resulting in the increase in the number and volume of lipid droplets. After the treatment with artesunate (purchased from Jiangsu Zhangjiagang Weisheng Biomedical Co., Ltd), the content of triglyceride (TG) in HepG2 liver cancer cells significantly decreased (as shown in FIG. 1), and the number of large lipid droplets decreased (as shown in FIGS. 2A-2B). FFA was not added to the medium of the control group, while FFA was added to the mediums of the model group and artesunate group. The addition amount is shown in FIG. 1, The formation of large lipid droplets in the model group is an important marker of steatosis in liver cells.

Experimental methods: HepG-2 liver cancer cells with passage times less than 20 times were selected and inoculated into a 12-well plate at a density of 3×10⁵ cells/mL. Drug treatment was performed after inoculating for 24 h, and the cells were divided into 3 groups with 4 wells in each group. A complete medium containing 1% bovine serum albumin (BSA) was used as the control group. A complete medium containing 1% BSA and 1 mM/L FFA was used as the high-fat model group. A complete medium containing 1% BSA, 1 mM/L FFA, and 5 μM/L, artesunate was used as the artesunate group. After being treated for 24 h, the cells were collected, and RNA was extracted and reverse-transcribed into cDNA. Quantitative real-time PCR (qRT-PCR) was used to detect the expression of CIDE series genes. AMPK signaling pathway-related genes, and PPAR signaling pathway-related genes in the three groups of cells. Primer sequences for the related genes are shown in Table 1.

TABLE 1
Primers for qRT-PCR reaction (human)

| | Forward primer | Reverse primer |
|---|---|---|
| Primer | (5′-3′) | (5′-3′) |
| GAPDH | TCCCTCAAGATTGTCAGCAA | AGATCCACAACGGATACA |
| | (SEQ ID NO: 1) | (SEQ ID NO: 2) |
| AMPKα | TCCTGGAGAAAGATGGCGAC | TTCACTTTGCCGAAGGTGCC |
| | (SEQ ID NO: 3) | (SEQ ID NO: 4) |
| ACC1 | TGAAGGCTGTGGTGATGGAT | CCGTAGTGGTTGAGGTTGGA |
| | (SEQ ID NO: 5) | (SEQ ID NO: 6) |
| SCD1 | GCGATATGCTGTGGTGCTTA | GGAGTGGTGGTAGTTGTGGA |
| | ATGC (SEQ ID NO: 7) | AGC (SEQ ID NO: 8) |
| FAS | TACATCGACTGCATCAGGCA | GATACTTTCCCGTCGCA |
| | (SEQ ID NO: 9) | (SEQ ID NO: 10) |
| CD36 | ACTGCAGTGTAGGACTTTCC | CCGGTCACAGCCCATTTTTC |
| | TG (SEQ ID NO: 11) | (SEQ ID NO: 12) |
| CPT-1 | GCTGATGACGGCTATGGTGT | GTCCATGGTCTCCTCCAAGG |
| | (SEQ ID NO: 13) | (SEQ ID NO: 14) |
| PPARγ | CGAGAGTCAGCCTTTAACGA | AGATGCAGGCTCCACTTTGA |
| | (SEQ ID NO: 15) | (SEQ ID NO: 16) |
| SREBP1c | GGCACGGGAGGATGGACT | GCTTCTTTGCTGTGAGATGA |
| | (SEQ ID NO: 17) | CC (SEQ ID NO: 18) |
| PPARα | TCCTCGGTGACTTATCCTGT | GCGTGGACTCCGTAATGATA |
| | GGTC (SEQ ID NO: 19) | GCC (SEQ ID NO: 20) |
| SIRT1 | GCTCCTACTGGCCTGAGGTT | TGCTGCGGAAAGAACAAGGC |
| | G (SEQ ID NO: 21) | (SEQ ID NO: 22) |
| Cide-A | GGAAAGGGCTTGGTGGTACA | TGGCTCTGACACATGCACAC |
| | (SEQ ID NO: 23) | (SEQ ID NO: 24) |
| Cide-B | CAGACCCCACCTCAAACACA | AATCCCTGCTTTCCTGCCAA |
| | (SEQ ID NO: 25) | (SEQ ID NO: 26) |
| Cide-C | GCCATGAAGTCCCTTAGCCT | GGCTTCAGGGTTCCTAGTCT |
| | (SEQ ID NO: 27) | (SEQ ID NO: 28) |

1) Expression opf Cell-Death-Inducing DNA-Fragmentation-Factor-Like Effector (CIDE) Series Genes Associated with Lipid Droplets in HepG2 Liver Cancer Cells

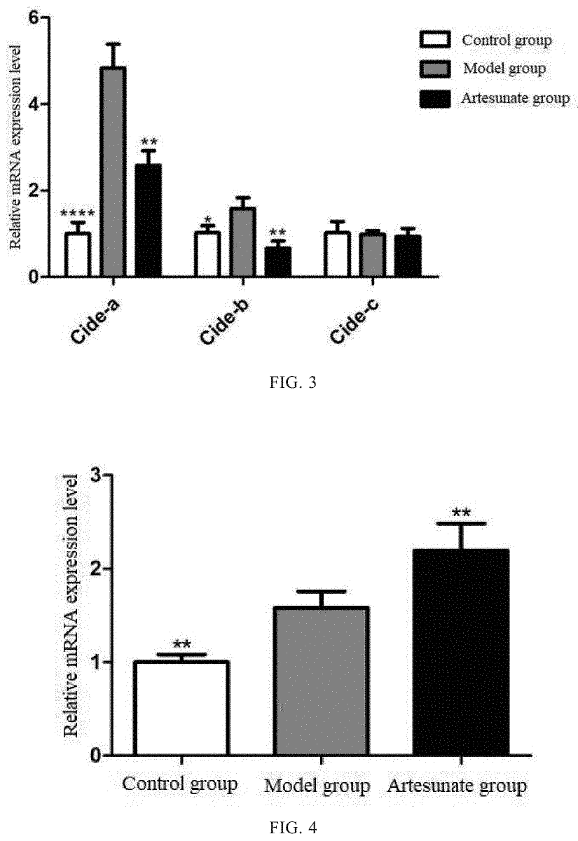

CIDE series genes include the Cide-a gene, the Cide-b gene, and the Cide-c gene. The expression. level of these genes is positively correlated with the size of lipid droplets. By testing the mRNA expression levels of the Cide-a gene, Cide-b gene, and Cide-c gene in the control group, model group, and artesunate group, it was found that the mRNA expression of Cide-a gene and Cide-b gene in the artesunate group were significantly lower than those in the model group, as shown in FIG. 3. Artesunate can reduce the expression of CIDE series genes, suggesting that artesunate can reduce the size of lipid droplets by increasing fatty acid β-oxidation. FIG. 3 shows, assuming the mRNA expression of CIDE series genes in the control group as 1, the relative mRNA expression in other groups compared with the control group.

2) Verification of AMPK Signaling Pathway-Related Gene Expression in Cells

Artesunate can act as an AMPK activator to some extent. Therefore, after artesunate treatment, the mRNA level of AMPKα (PRKAA1) in HepG2 cells in the artesunate group was significantly up-regulated compared with that in the model group (p<0.01), as shown in FIG. 4.

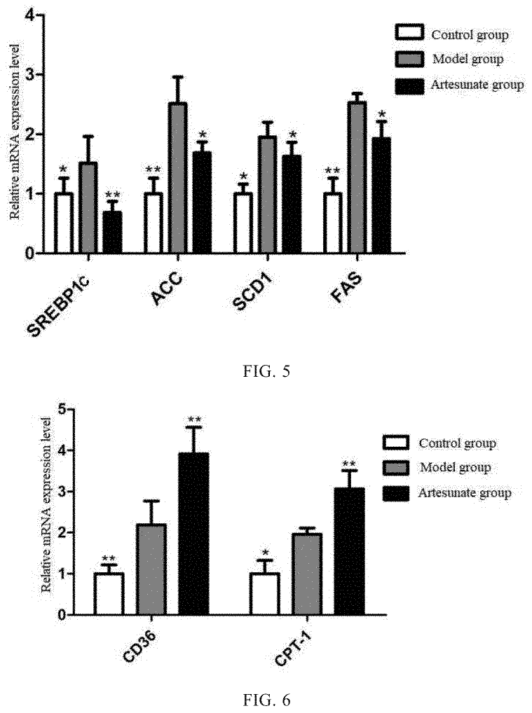

The effect of artesunate on AMPK-SREBP1c-ACC/SCD1/FAS signaling pathways is shown in FIG. 5, SREBP1c-ACC/SCD1/FAS are genes related to intracellular fatty acid synthesis; artesunate can reduce the expression of those genes related to the pathways, reduce the synthesis of fatty acids in cells, activate AMPK genes, and increase the fatty acid β-oxidation capacity in liver cells.

Effects of artesunate on AMPK-CD36-CPT1 pathways showed that: After artesunate treatment, the mRNA level of carnitine palmitoyl transferase 1 (CPT-1) in HepG2 cells in the artesunate group was significantly up-regulated compared with that in the model group (p<0.01), as shown in FIG. 6. The increase in the expression of CPT-1 can accelerate the transport of fatty acids into mitochondria for oxidative degradation.

3) Expression Levels of PPAR Signaling Pathway-Related Gene in Cells

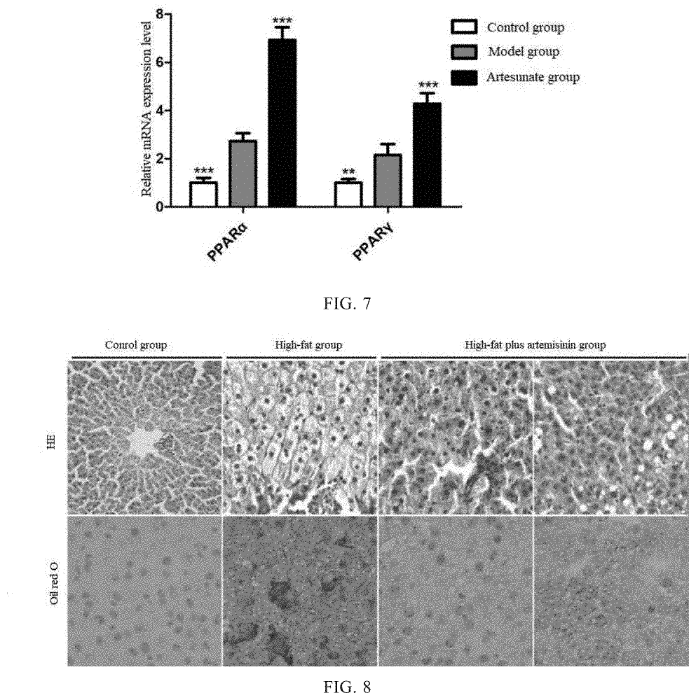

After artesunate treatment, the mRNA levels of PPARct. and PPARy in HepG2 cells in the artesunate group were significantly up-regulated compared with those in the model group (p<0.001), as shown in FIG. 7. PPAR is a key gene affecting fatty acid β-oxidation, and the increase in the expression thereof indicates that the fatty acid β-oxidation is accelerated in cells.

### Embodiment 2

C57 mice were selected and fed with feedstuffs that were added with 10% lard and 5% fructose. After 12 weeks, the mice suffered from liver metabolism disruption, generated insulin resistance, and were stuck in mitochondrial dysfunction in liver cells, thus resulting in a decrease in fatty acid β-oxidation capacity and fat accumulation in the liver, and inflammation and fibrosis were produced. Artemisinin was given at a dosage of 100 mg/kg from the 13th week as a treatment for 12 weeks. At the end of the experiment, the mice were dissected to obtain livers. A part of the livers was subjected to HE staining and Oli red O staining to quantify the liver lipids and conduct a pathological scoring of liver steatosis of the mice based on the steatosis activity fibrosis (SAF) scoring system. The results showed that there were obvious fat blebs in the mice's livers after high-fat induction, and large red areas were observed after the Oil red O staining. The fat blebs and Oil red O staining areas in the livers of the mice treated with artemisinin were both closer to those of the mice in the normal group, indicating that artemisinin intervention could significantly improve liver steatosis induced by a high-fat diet, as shown in FIG. 8.

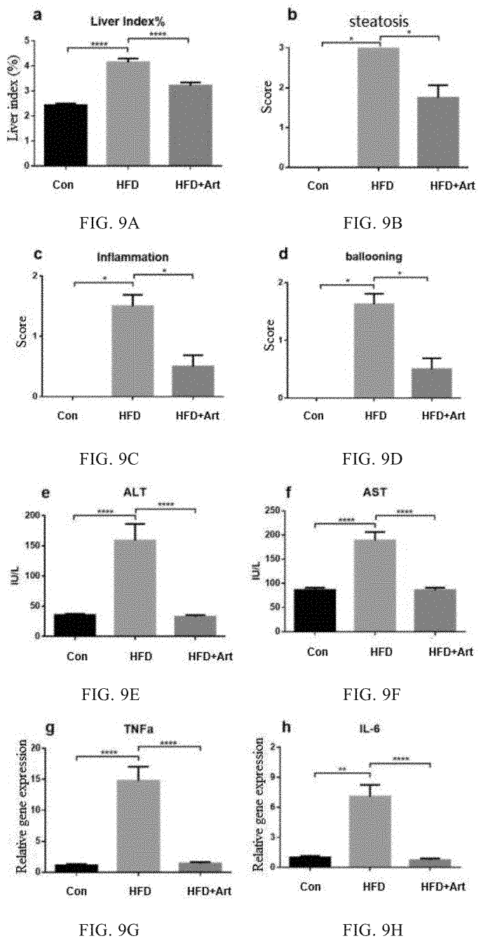

The serum of each group of mice mentioned above was taken and subjected to serum enzyme detection. The results showed that artemisinin could significantly reduce the elevation of ALT and AST levels in the serum induced by the high-fat diet. Pathological scores based on the SAF scoring system also showed that artemisinin intervention significantly reduced the scores of lobular inflammation and ballooning of liver cells, as shown in FIGS. 9A-9H.

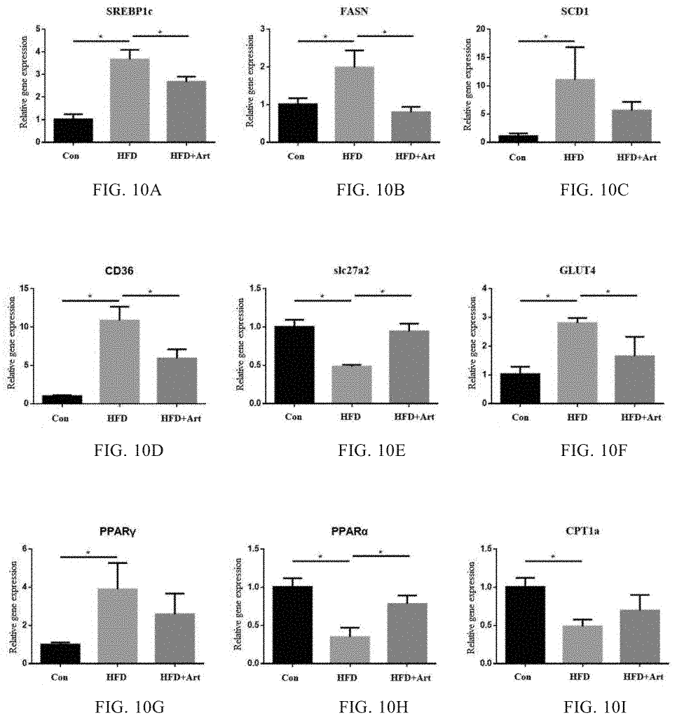

A part of the livers of the mice in each group mentioned above was taken, RNA and protein were extracted, and the study of the expression of related genes and proteins was conducted to detect the expression levels of lipid metabolism-related genes in liver tissue. The results showed that the upstream gene expression levels of genes related to lipid synthesis were inhibited in mice treated with artemisinin on the high-fat and high-sugar diet. The results suggest that artemisinin can reduce the lipid content in the liver of mice induced by the high-fat and high-sugar diet by reducing the expression of genes related to lipid synthesis, as shown in FIGS. 10A-10I. The primer sequences of the genes are shown in Table 2.

TABLE 2
Primers for qRT-PCR reaction (mouse)

| | Forward primer | Reverse primer |
|---|---|---|
| Primer | (5′-3′) | (5′-3′) |
| GAPDH | GTTGTCTCCTGCGACTTCA | GCCCCTCCTGTTATTATGG |
| | (SEQ ID NO: 29) | (SEQ ID NO: 30) |
| Slc27a2 | ATGGGACAAGCCTTGCTATG | CTCAGTCATGGGCACAAATG |
| | (SEQ ID NO: 31) | (SEQ ID NO: 32) |
| Glut4 | GAAGGGTGCTAAACCCGAAA | TCTGCTCCCTATATCCGTTC |
| | (SEQ ID NO: 33) | TT (SEQ ID NO: 34) |
| SCD1 | TGGGGCTGCTAATCTCTGGG | GGCTTTATCTCTGGGGTGGG |
| | TGT (SEQ ID NO: 35) | TTTG(SEQ ID NO: 36) |
| FASN | AAGTTGCCCGAGTCAGAGAA | CGTCGAACTTGGAGAGATCC |
| | (SEQ ID NO: 37) | (SEQ ID NO.38) |
| CD36 | TGGTCAAGCAGCTAGAAA | CCCAGTCTCATTTAGCCAC |
| | (SEQ ID NO: 39) | (SEQ ID NO: 40) |
| CPT-1 | GACTCCGCTCGCTCATTCC | GGCAGATCTGTTTGAGGGCT |
| | (SEQ ID NO: 41) | (SEQ ID NO: 42) |
| PPARγ | GTGCCAGTTTCGATCCGTAG | GGCCAGCATCGTGTAGATGA |
| | A (SEQ ID NO: 43) | (SEQ ID NO: 44) |
| SRPBP1c | CTGGTGAGTGGAGGGACCAT | TGCTGCAAGAAGCGGATGTA |
| | (SEQ ID NO: 45) | (SEQ ID NO: 46) |
| PPARα | TGGATGGATTAGATTGGACT | TGTTGATGAGCCTGACTTCA |
| | G (SEQ ID NO: 47) | T (SEQ ID NO: 48) |

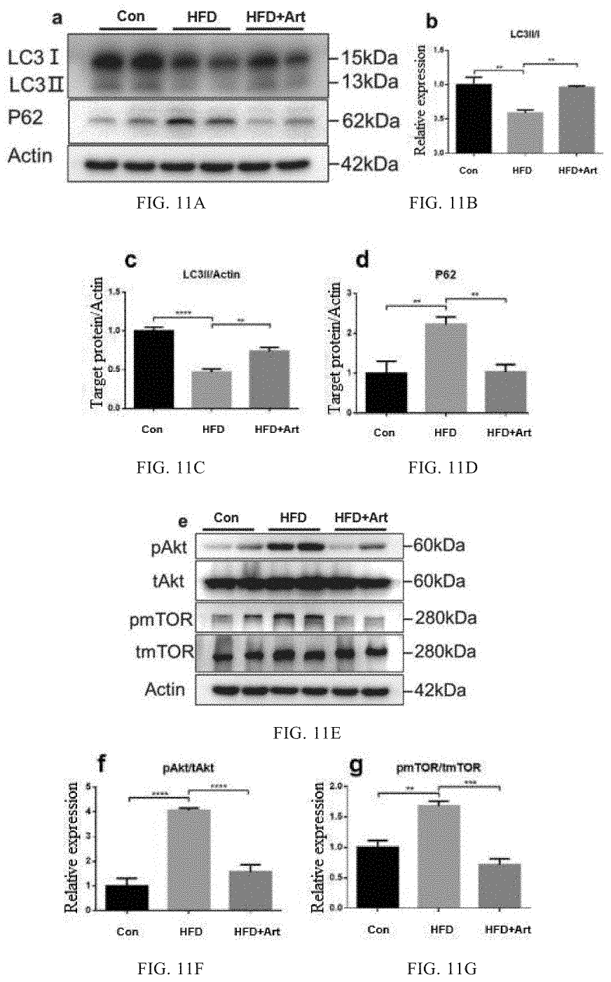

Target of rapamycin (TOR) is a key protein in the control of autophagy, which can sense various change signals of cells and strengthen or reduce the occurrence level of autophagy. The above studies on the expression of mice liver proteins revealed that TOR was abnormally activated in the liver of mice having a high-fat and high-sugar diet, and mitopha.gy was significantly inhibited. When the mice fed with a high-fat and high-sugar diet were subjected to the artemisinin intervention, the mice could effectively resist the mitochondrial dysfunction and the decrease in the autophagy level caused by the high-fat and high-sugar diet, thereby reducing steatosis and inflammation in the liver, as shown in FIGS. 11A-11G. LC3II/Actin is marked in FIG. 11C, indicating that the expression level of LC3II protein in the control group (Con), high-fat group (HFD), and high-fat plus artemisinin group (HFD+ART) was homogenized by the ratio of the expression level of LC3II protein to that of the reference protein Actin.

The animal experiment mentioned above showed that the addition of high-fat and high-sugar in the diet without adding additional chemical inducers can simulate the metabolic dysfunction caused by a long-term poor diet after induction up to 12 weeks, especially mitochondrial dysfunction, decrease in fatty acid β-oxidation capacity, thereby leading to fat accumulation in the liver and secondary inflammation and fibrosis. Artemisinin treatment can significantly improve liver steatosis and inflammation in mice. As the center of energy metabolism in eukaryotic cells, the homeostasis of mitochondria in cells plays an essential role in the normal operation of cells. When mitochondria are senescent or dysfunctional, to maintain the normal operation of cells, on the one hand, the cells will clear the abnormal mitochondria through autophagy, and this mitochondrial clearance mechanism is also commonly defined as mitophagy. On the other hand, if mitochondria with abnormal function cannot be removed in time, that is, when the function of mitopha.gy is defective, a large number of mitochondrial free radicals derived from mitochondria will be produced, leading to inflammation. The induction of high-fat and high-sugar can increase mitochondrial pressure levels, causing swelling and functional damage. Mitophagy is the targeted phagocytosis and destruction of mitochondria. by cellular autophagy devices, which is generally considered to be the main mechanism of mitochondrial quality control. Artemisinin treatment can restore the level of mitophagy in mice induced by a high-fat and high-sugar diet to maintain the quantity and quality of mitochondria in the liver of mice, restore the function of mitochondria, and inhibit the occurrence of liver fatty lesions and inflammation.

TABLE 3
English abbreviations

| English | |
|---|---|
| abbreviation | Full English name |
| AMPK | Adenosine 5′-monophosphate (AMP)-activated |
| | protein kinase |
| PPAR | Peroxisome proliferator-activated receptor |
| FFA | Free fatty acid |
| SREBP-1c | Sterol regulatory element-binding proteins-1c |
| ABCA1 | ATP-binding cassette transporter A1 |
| ABCG1 | ATP-binding cassette transporter G1 |
| LXRs | Liver X receptors |
| HDL-C | High density lipoprotein cholesterol |
| CPT-1 | Carnitine palmitoyl transferase 1 |
| SIRT1 | Silent mating type information regulation 2 homolog l |
| CD36 | cluster of differentiation 36 |
| acyl-CoA | Acyl-coenzyme A |
| FAS | Fatty acid synthase |
| ACC | Acetyl coA carboxylase |
| malonyl | Malonyl coenzyme A |
| CoA | |
| SCD1 | Stearoyl-coA desaturase-1 |
| ACSL1 | Long chain fatty acid-CoA ligase 1 |
| PGC-1α | peroxisome proliferator-activated receptor γ |
| | coactivator 1α |
| mTOR | mammalian target of rapamycin |
| TC | Total Cholesterol |
| TG | Triglyceride |
| CYP7A1 | cholesterol 7-alpha hydroxy-lase |
| ALT | Alanine aminotransferase |
| AST | Aspartate aminotransferase |
| Slc27a2 | solute carrier family 27 (fatty acid transporter), |
| | member 2 |
| GLU4 | solute carrier family 2 (facilitated glucose transporter), |
| | member 4 |

Preferred specific embodiments of the present invention are described in detail above. It should be understood that the ordinary skilled in the art is capable of performing numerous modifications and variations based on the concept of the present invention without creative labor. Therefore, any technical solution which can be obtained by those skilled in the art based on the concept of the present invention and the prior art by logical analysis, reasoning, or limited experiments shall fall within the scope of protection as determined by the claims.

What is claimed is:

1. A method of an application of a compound using intra-cyclic peroxo-bridged sesquiterpenes as a parent nucleus in metabolism-related fatty liver diseases, wherein the compound using the intra-cyclic peroxo-bridged sesquiterpenes as the parent nucleus has a structure shown in Formula I: 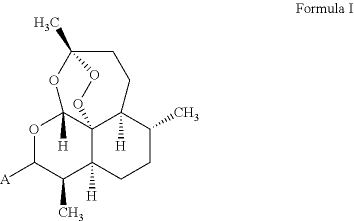
wherein A denotes O, —OH, —OCH₃, —OCH₂CH₃, or —OC₄H₅O₃; the compound using the intra-cyclic peroxo-bridged sesquiterpenes as the parent nucleus is configured for preparing a drug or a composition for treating the metabolism-related fatty liver diseases.

2. The method according to claim 1, wherein when A is the —OC₄H₅O₃, the compound using the intra-cyclic peroxo-bridged sesquiterpenes as the parent nucleus is artesunate.

3. The method according to claim 1, wherein the compound using the intra-cyclic peroxo-bridged sesquiterpenes as the parent nucleus has a function of regulating mitochondria in liver cells.

4. The method according to claim 3, wherein the function of regulating the mitochondria in the liver cells is to enhance a fatty acid β-oxidation capacity of the mitochondria.

5. The method according to claim 1, wherein the metabolism-related fatty liver diseases comprise liver lesions caused by a mitochondrial dysfunction and a decreased fatty acid n-oxidation capacity; the liver lesions comprise liver steatosis, inflammation, and fibrosis.

6. A drug comprising the compound with the structure shown in the Formula I according to claim 1 for a prevention or a treatment of the metabolism-related fatty liver diseases.

7. A composition for a prevention or a treatment of the metabolism-related fatty liver diseases comprising the compound with the structure shown in the Formula I according to claim 1 or a pharmaceutically acceptable salt of the compound, as an active ingredient.

8. The composition according to claim 7, further comprising one or more carriers, excipients, or diluents pharmaceutically acceptable.

9. A method of an application of the compound with the structure shown in the Formula I according to claim 1 or a pharmaceutical salt of the compound in a preparation of products for a prevention or a treatment of the metabolism-related fatty liver diseases.

10. The method according to claim 9, the metabolism-related fatty liver diseases are liver lesions caused by a mitochondrial dysfunction and a decreased fatty acid β-oxidation capacity; the liver lesions comprise liver steatosis, inflammation, and fibrosis.
